SEQUENCE LISTING (16 sequences)

SEQ ID NO: 1 - DNA, 64 bp, Staphylococcus aureus
  misc_feature: agr gene of CA-MRSA MW2
gttataacaatttttataataatttttctctattttaaaattagactatattctgtatttttag

SEQ ID NO: 2 - DNA, 142 bp, Staphylococcus aureus
  misc_feature: arg gene of CA-MRSA MW2
aattgttcaagtttcattaatgttctttatatctgcatttattagtggaataagatacaaaaaatcagattatatatacattattggaatagtgttatcttcagtatatttctttgacaaaatcggaagtatttcactagtt

SEQ ID NO: 3 - DNA, 65 bp, Staphylococcus aureus
  misc_feature: agr gene of CA-MRSA USA300
atttatacttttacctttagtaatagtaaattttcatattaactttttaattatgattattttaa

SEQ ID NO: 4 - DNA, 66 bp, Staphylococcus aureus
  misc_feature: agr gene of CA-MRSA USA300
tattcttttatttttattggtatcactatatttttaagtatattaacatttgttatttctcaattt

SEQ ID NO: 5 - DNA, 20 bp, Staphylococcus aureus
  misc_feature: forward primer specific for a target DNA sequence of the agr gene of CA-MRSA MW2
tttttataataatttttctc

SEQ ID NO: 6 - DNA, 20 bp, Staphylococcus aureus
  misc_feature: reverse primer specific for a target DNA sequence of the agr gene of CA-MRSA MW2
ctaaaaatacagaatatagt

SEQ ID NO: 7 - DNA, 19 bp, Staphylococcus aureus
  misc_feature: forward primer specific for a target DNA sequence of the agr gene of CA-MRSA MW2
tttattagtggaataagat

SEQ ID NO: 8 - DNA, 19 bp, Staphylococcus aureus
  misc_feature: reverse primer specific for a target DNA sequence of the agr gene of CA-MRSA MW2
ttgttataacaactagtga

SEQ ID NO: 9 - DNA, 19 bp, Staphylococcus aureus
  misc_feature: forward primer specific for a target DNA sequence of the agr gene of CA-MRSA USA300
atttatacttttaccttta

SEQ ID NO: 10 - DNA, 20 bp, Staphylococcus aureus
  misc_feature: reverse primer specific for a target DNA sequence of the agr gene of CA-MRSA USA300
ttaaaataatcataattaaa

SEQ ID NO: 11 - DNA, 21 bp, Staphylococcus aureus
  misc_feature: forward primer specific for a target DNA sequence of the agr gene of CA-MRSA USA300
tattcttttatttttattggt

SEQ ID NO: 12 - DNA, 22 bp, Staphylococcus aureus
  misc_feature: reverse primer specific for a target DNA sequence of the agr gene of CA-MRSA USA300
aaattgagaaataacaaatgtt

SEQ ID NO: 13 - DNA, 20 bp, Staphylococcus aureus
  misc_feature: forward primer specific for a target DNA sequence of the pvl gene of CA-MRSA
caggtgtgatatgttgagct

SEQ ID NO: 14 - DNA, 21 bp, Staphylococcus aureus
  misc_feature: reverse primer specific for a target DNA sequence of the pvl gene of CA-MRSA
tatttattcgtacaaagtcca

SEQ ID NO: 15 - DNA, 20 bp, Staphylococcus aureus
  misc_feature: forward primer specific for a target DNA sequence of the spa gene of CA-MRSA
tcaacaacaagttcttgacc

SEQ ID NO: 16 - DNA, 21 bp, Staphylococcus aureus
  misc_feature: reverse primer specific for a target DNA sequence of the spa gene of CA-MRSA
acagtaaatgacattgcaaaa

(19) United States
(12) Patent Application Publication
(10) Pub. No.: US 2010/0152432 A1
(43) Pub. Date: Jun. 17, 2010

(54) DNA sequences and primers for identifying methicillin-resistent Staphylococcus aureus MW2 and USA300 strains

(76) Inventor: Ling-Chuan Chen WU, Lawrenceville, NJ (US)

Correspondence Address: Lu & Associates, P.C., P.O. Box 1380, HAVERTOWN, PA, 19083, US

(21) Appl. No.: 12/265,298

(22) Filed: Nov. 5, 2008

Related U.S. Application Data
(60) Provisional application No. 60/987,548, filed on Nov. 13, 2007.

(51) Int. Cl. C07H 21/04

(52) U.S. Cl. 536/24.33; 536/23.1

(57) ABSTRACT

The present invention relates to unique agr gene sequences and primer sets for identifying CA-MRSA MW2 or CA-MRSA USA300 strains in clinical samples from various sources. More specifically, this invention provides fluorescent labeled primers which are designed to amplify 1) two unique agr gene sequences from each strain, 2) a partial spa gene and 3) a partial pvl gene. These primer sets can be packaged into one multiplex PCR kit for identifying MRSA strains that are most virulent and pathogenic.

### CROSS REFERENCE APPLICATION

This application claims priority from U.S. Provisional Patent Application No. 60/987,548 filed on Nov. 13, 2007.

### FIELD OF THE INVENTION

The present invention provides unique DNA sequences and primers for detecting highly virulent and invasive methicillin-resistent Staphylococcus aureus (hereinafter referred to as MRSA) MW2 and USA300 strains. More specifically, this invention provides sets of primer to amplify unique agr gene sequences in either MRSA MW2 or USA300 strains. Together with the primer sets that amplify a partial pvl gene and a partial spa gene, this invention provides molecular biological tools that can be packaged into one testing kit for identifying MRSA strains that are most virulent and pathogenic.

### BACKGROUND OF THE INVENTION

S. aureus is a potentially pathogenic bacterium that causes a broad spectrum of conditions in human beings, ranging from carrier state to mild skin diseases and life-threatening invasive infection. The carrier rate of S. aureus world-wide is about 30 percent. In the United States hospitals, it was estimated that approximately 500,000 patients contracted an infection per year. Those who infected with S. aureus stay 3 times longer in the hospital and are 5 times more likely to die comparing to patients who are not infected. Furthermore, S. aureus isolated in the patient's blood stream were associated with 20-40 percent fatality.

The infectivity and the severity of the infection caused by S. aureus are mainly determined by the patient's condition as well as by genetic background of the bacteria. The most pathogenic strains carry Panton-Valentine leukocidin (PVL) to destroy neutrophiles; staphylococcal protein A (SpA) to induce the production of proinflammatory chemokine and cytokine; various superantigens and enterotoxins to trigger an overstimulation of the immune system; and phenol-soluble modulin (PSM) peptides to lyse human neutrophiles (Wang et al., 2007, Nature Medicine 13:1510-1514, published online 11 Nov. 2007). Although all sequenced S. aureus strains contain gene encoded for PSMs, their expression is tightly controlled by the agr quorum-sensing system (Wang et al., 2007, Nature Medicine 13:1510-1514, published online 11 Nov. 2007). S. aureus strains harbors these virulence genes are potentially infectious to healthy people in all age groups without risk factors. Although the majority of cases presented are primary skin infections, these strains are highly contagious. They have the potential to quickly progress to life-threatening infections such as necrotizing pneumonia, meningitis, and sepsis in healthy, young, immuno-competent individuals.

The new emerging community-acquired methicillin-resistant S. aureus (hereinafter referred to as CA-MRSA) that derived from the acquisition of methicillin-resistant gene cassettes (SCCmec) VI or V in a virulent S. aureus background has become the most important public health threat since early twenties. Outbreaks in households, day-care center, school and prison have been reported in recent years. It affects a large population in a very short period of time, and accounted for 98 percent of overall S. aureus infections currently. With the growing colonization rate of 0.8 percent in 2001 to 9.5 percent in 2005, CA-MRSA is the most common pathogen cultured in ER skin infections now. Approximately 50 percent of ER visit with skin problems are due to CA-MRSA, while secondary necrotizing pneumonia caused by CA-MRSA had also been reported in the 2006 flu season.

In contrast to the virulent S. aureus, the so-called hospital-acquired methicillin-resistant S. aureus (HA-MRSA) do not carry most of the virulence factors including PVL. The HA-MRSA that established and circulated in most of the US hospitals for the past ten to twenty years affected peoples with risk factors, such as prolong hospital stay, transplantation, catheterization, surgery, antibiotic exposure, etc. These strains rarely infect health care professionals whom, however, can be the carriers that transmit the bacteria from patient to patient. Without the virulence genes, the HA-MRSA enters the human's body and becomes systemic infections through the cuts, wounds, and the inserted catheter tubings and devices. The affected groups are often elderly people, especially those living in nursing home and long-term care facilities. The major problem associated with HA-MRSA is the limited treatment options owing to the existence of the methicillin-resistant gene cassettes carried by HA-MRSA (SCCmec I-III) which also carry multiple non-beta-lactam antibiotic-resistant determinants. For HA-MRSA, the only remaining antibiotics are vancomycin, linezolid, quinupristin and dalfopristin.

Unlike HA-MRSA isolates, CA-MRSA isolates from patients without known MRSA risk factors are generally resistant to fewer non-beta-lactam antibiotics. They grow significantly faster than the nosocomial strains and can be highly virulent to cause serious and often fatal disease in otherwise immunocompetent individuals. The evolution of CA-MRSA is believed to be a recent event due to the acquisition of a novel SCCmec IV (or V) cassette with methicillin-resistant gene by an otherwise susceptible S. aureus. Unlike SCCmec I-III, the characteristic cassettes carried by CA-MRSA, SCCmec IV (or V) is smaller in size and do not carry multiple non-beta-lactam antibiotic-resistant determinants. Depending on the severity of the infection and the local antibiogram, the physicians have choices of antibiotics, including clindamycin, trimethoprim-sulfamethoxazole, doxycycline, minocycline, and rifampin, while the vancomycin and other newer antibiotics can be preserved as the last resort for treating CA-MRSA.

Various SCCmec typing methods that based on amplifying different sequences of MRSA have been developed previously. For example, the amplification of SCCmec right extremity junction sequences for the types i, ii, iii, iv, v, vii (Huletsky et al., 2004, J. Clin. Microbiol. 42:1875-1884) and types xi, xii, xiii, xiv, xv, xvi, xvii, xviii, xix, and xx (Huletsky et al., 2007, United State Patent Application NO. 20070082340); and the amplification of mecA internal sequence and a chromosomal DNA surrounding the integrated mecA for differentiating MRSA and methicillin-resistant coagulase-negative staphylococci (Hiramatsu et al., 2000, U.S. Pat. No. 6,156,507). Moreover, Matsunaga et al. described a method and kit that includes amplifying both mecA and spa genes to distinguish MRSA from methicillin-resistant coagulase-negative staphylococci (Matsunaga et al., 1997, U.S. Pat. No. 5,702,895). However, none of these above mentioned methods include target genes and primer sequences that can indicate the invasiveness and infectivity of a MRSA strain in one multiplex PCR.

MRSA is conventionally identified by culturing bacteria isolated from clinical specimens and checking for the presence for drug sensitivity in most of the hospital or clinical laboratory in the United States. It is time consuming, requiring one day for cultivation and another day for drug sensitivity testing. Furthermore, the tests do not offer any information about the infectivity or the invasiveness of the strains. The time gap plus the lack of virulence indicators will put the decision making, such as whether to promptly triage the infected patient in isolation, on hold and uncertain. A quick test that can be done on site when receiving a patient in a hospital/long term care/nursing home/prison will be greatly helpful and cost-effective, if the test can also indicate the invasiveness of the strain. Depending on test results, the affected patient can be promptly confined to an isolation room as needed and a highly stringent infection control measure can be applied to prevent further spread of MRSA to other people. In addition, the virulence indicators are very useful in predicting the disease progression, the outcome, and the infectivity to other healthy person. The positive test results justify the need of an aggressive antibiotic treatment followed by continuing monitoring the treatment response and the total eradication of the infection.

To enable a timely response, I invented primer sets which can be used to amplify unique agr gene sequences in either CA-MRSA MW2 (USA400) (Baba et al., 2002, Lancet 359:1819-1827) or CA-MRSA USA300 (LAC) (Diep et al., 2006, Lancet 367: 731-739) strains. Together with the primer sets for amplifying partial pvl gene and spa gene, these target sequences provide tools for one-step identification of the most virulent and pathogenic S. aureus strains in United States.

The agr gene locus, which comprises two divergent operons from promoters P2 and P3, was initially described in S. aureus as an element controlling the production of exoproteins implicated in virulence. The P2 operon includes four genes, of which two encode elements of density-sensing cassette, agrD encodes the precursor of the autoinducing peptide (AIP), and agrB, whose product is probably involved in processing and/or secretion of AIP (Dufour et al., 2002, J. Bact. 184:1180-1186). The two component sensory transduction system is comprised of AgrC, the membrane sensor, and AgrA, the response regulator. In brief, the AIP derived from AgrD by the action of AgrB interacts with AgrC in the membrane to activate AgrA, which upregulates transcription both from P2, amplifying the response, and from P3, initiating the production of a novel effector: RNAIII. In S. aureus, delta-hemolysin is the only translation product of RNA III (Dufour et al., J. Bact. 2002, 184:1180-1186).

Although the PSM genes are present in all sequenced S. aureus strains, Wang, et al. (Wang et al., 2007, Nature Medicine 13:1510-1514, published online 11 Nov. 2007) detected much higher in vitro PSM production in the most prevalent CA-MRSA, including MW2 and USA300, when compared to HA-MRSA. Furthermore, all S. aureus PSMs are tightly controlled by the agr quorum-sensing system, and the strain-to-strain differences in PSM production seem to be caused in part by differential agr activity.

The pvl gene encodes PVL which is the major virulence factors in CA-MRSA and certain MSSA strain. The pvl locus encodes two exotoxins—LukS-PV and LukF-PV which act together as subunits, and form a ring with a central pore in the membrane of white blood cells and thus destroy neutrophiles. Although, the predominant CA-MRSA studied so far in the United States has shown a strong association with PVL positively, a recent investigation in Ireland reveled that pvl gene is a poor marker for CA-MRSA, and the presence of pvl gene cannot be used as a sole marker for CA-MRSA (Rossney et al., 2007, J. Clin. Microbiol. 45: 2554-2563).

The spa gene encodes the SpA which is only presented in all S. aureus, but not in Staphylococcus epidermidis or other coagulase-negative Staphylococcus. PVL increases the expression of SpA, which, in turn, binds to tumor necrosis factor receptor and triggers the overproduction of proinflammatory chemokine and cytokine Strains that are spa and pvl positive are most virulent and often associate with fatal necrotizing pneumonia.

This invention utilizes an extensive DNA databases and queries collected and provided by the National Center for Biotechnology Information (hereinafter referred to as NCBI), National Institute of Health, to search unique and non-homologous agr DNA sequences in either CA-MRSA MW2 or CA-MRSA USA300 strains, such that the amplification of these regions represent only CA-MRSA MW2 or CA-MRSA USA300 strains. The amplification of these unique and non-homologous agr DNA sequences, together with the additional primer sets for amplifying spa and pvl genes, provide tools for a quick test with the discriminatory power to identify the most predominant, virulent and invasive CA-MRSA in the United States.

### BRIEF SUMMARY OF THE INVENTION

This invention describes unique DNA sequences and primer sets to identify the presence of proper agr genetic background in CA-MRSA MW2 or CA-MRSA USA300 strains. Fluorescent labeled primers which are designed to amplify and detect 1) two unique agr gene sequences from each strain, 2) a partial spa gene and 3) a partial pvl gene are presented. The amplification of these target sequences provides a simple and rapid identification of the most virulent/invasive MRSA strains in the United States.

### BRIEF DESCRIPTION OF THE DRAWING

FIG. 1 depicts the flow chart of searching and selecting unique agr gene sequences in CA-MRSA MW2 or USA300 strains for PCR amplification and the designing and labeling of the primers for amplifying these unique agr gene sequences.

FIG. 2 depicts the flow chart of searching and selecting pvl gene sequences in CA-MRSA MW2 or USA300 strains for PCR amplification and the designing and labeling of the primers for amplifying these pvl sequences.

FIG. 3 depicts the flow chart of searching and selecting spa gene sequences in CA-MRSA MW2 or USA300 strains for PCR amplification and the designing and labeling of the primers for amplifying these spa sequences.

### DETAILED DESCRIPTION OF THE INVENTION

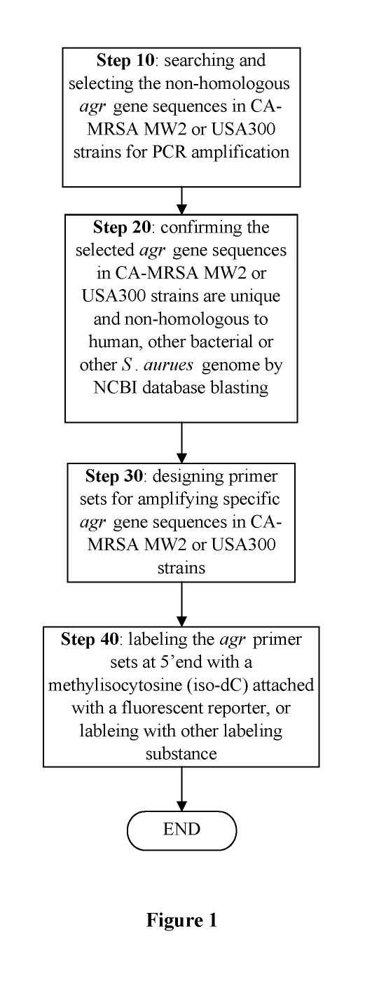

In the first dimension, this invention provides unique agr gene sequences that can represent only CA-MRSA MW2 or USA300 strains. Amplification of these agr gene sequences by the primer sets specifically designed and labeled provides identifiers to discriminate other staphylococci in clinical samples. A flow chart of steps that describe the invention of proper agr gene and primer sequences is depicted in FIG. 1. Detailed description of FIG. 1 is presented by steps as follows.

FIG. 1 Step 10: Searching and selecting the non-homologous agr gene sequences in CA-MRSA MW2 or USA300 strains for multiplex PCR amplification

This invention utilizes DNA databases and queries collected and provided by NCBI, National Institute of Health, to search the whole genome of CA-MRSA MW2 strain. The search result yields one GenBank accession number BA000033, submitted by Baba et al. (Baba et al., 2002, Lancet 359:1819-1827). A 2760 base pair fragment, spanning agr locus (base pair number from 2108049 to 211088 (GenBank accession number NC_{—}003923.1) in CA-MRSA MW2 strain) was selected for aligning agr locus from CA-MRSA USA300 (GenBank accession number NC_{—}007793.1) and four agr alleles published by Jarraud et al. (Jarraud et al., 2002, Infection and Immunity 70:631-641). Each of the four agr alleles was designated agr-1ₛₐ (GenBank accession number X52543); agr-2ₛₐ (GenBank accession number AF001782); agr-3ₛₐ (GenBank accession number AF001783), and agr-4ₛₐ (GenBank accession number AF288215) (Jarraud et al., 2002, Infection and Immunity 70:631-641). The alignment was carried out by NCBI Blast 2 software (version LASTN 2.2.17 [Aug. 26, 2007]).

A 64 base pair DNA sequence located in agrC (from base pair number 2108961 to 2109024, GenBank accession number BA000033, SEQ ID NO:1) in CA-MRSA MW2 genome was found that is unique from the corresponding agrC sequence derived from each of the CA-MRSA USA300, agr-1ₛₐ, agr-2ₛₐ, agr-3ₛₐ, and agr-4ₛₐ.

The complete DNA sequence of SEQ ID NO: 1 is GTTATAACAATTTTTATAAT AATTTTTCTCTATTTTAAAATTAGACTATATTCTGTATTTTTAG.

Similarly, a 142 base pair DNA sequence also located in agrC (from base pair number 2108819 to 2108960, GenBank accession number BA000033, SEQ ID NO:2) in CA-MRSA MW2 genome was also found which was non-homologous to the corresponding agrC sequence derived from each of the CA-MRSA USA300, agr-1ₛₐ, agr-2ₛₐ, and agr-4ₛₐ. However, they are highly similar with the corresponding agr-3ₛₐ sequence, with only 2 mis-matched base pairs.

The complete DNA sequence of SEQ ID NO:2 is AATTGTTCAAGTTTCATTAA TGTTCTTTATATCTGCATTTATTAGTGGAATAAGATACAAAAAATCAGATTATATAT ACATTATTGGAATAGTGTTATCTTCAGTATATTTCTTTGACAAAATCGGAAGTATTTC ACTAGTT.

A similar search was carried out using a 5001 base pair sequence spanning the agr locus in CA-MRSA USA300 strain (from base pair number 2145000 to 2150000, GenBank accession number NC_{—}007793.1) to align each of the agr locus in the CA-MRSA MW2, agr-1ₛₐ, agr-2ₛₐ, agr-3ₛₐ, and agr-4ₛₐ. Each of the four agr alleles was designated agr-1ₛₐ (GenBank accession number X52543); agr-2ₛₐ (GenBank accession number AF001782); agr-3ₛₐ (GenBank accession number AF001783), and agr-4ₛₐ (GenBank accession number AF288215) (Jarraud et al., 2002, Infection and Immunity 70:631-641). The alignment was carried out by NCBI Blast 2 software (version LASTN 2.2.17 [Aug. 26, 2007]).

A 65 base pair DNA sequence located in agrB (from base pair number 2147214 to 2147278, GenBank accession number NC_{—}007793.1, SEQ ID NO:3) in CA-MRSA USA300 genome was found that is unique from the corresponding agrB sequence derived from each of the CA-MRSA MW2, agr-1ₛₐ, agr-2ₛₐ, agr-3ₛₐ, and agr-4.

The complete DNA sequence of SEQ ID NO:3 is ATTTATACTTTTACCTTTAG TAATAGTAAATTTTCATATTAACTTTTTAATTATGATTATTTTAA.

Similarly, a 66 base pair DNA sequence located in agrC (from base pair number 2148216 to 2148281, GenBank accession number NC_{—}007793.1, SEQ ID NO:4) in CA-MRSA USA300 genome was found which was non-homologous to the corresponding agrC sequence derived from each of the CA-MRSA MW2, agr-1ₛₐ, agr-2ₛₐ, agr-3ₛₐ, and agr-4ₛₐ.

The complete DNA sequence of SEQ ID NO:4 is TATTCTTTTATTTTTATTGG TATCACTATATTTTTAAGTATATTAACATTTGTTATTTCTCAATTT.

FIG. 1 Step 20: Confirming the selected agr gene sequences in CA-MRSA MW2 or CA-MRSA USA300 strains are unique and non-homologous to human, other bacterial or other S. aureus genome by NCBI database blasting

To confirm that each of the SEQ ID NO:1 to ID NO:4 is unique and therefore can be used in the PCR amplification products as diagnostic sequences among mixed specimens from human clinical samples, a confirmation test was performed. The confirmation test uses each of SEQ ID NO:1 to ID NO:4 as a query sequence in search for a homologous sequence among databases collected by NCBI GenBank in five categories: a) human genome sequence (Posted date: Apr. 16, 2008 7:40 PM); b) patent sequence (nucleotide sequences derived from the Patent Division of GenBank, Posted date: Aug. 3, 2008 4:57 AM); c) NCBI chromosome sequences (Posted date: Aug. 5, 2008 4:57 AM); d) NCBI chromosome sequence, under bacteria subdivision (Taxid: 2) (Posted date Posted date: Aug. 5, 2008 4:57 AM); and e) NCBI chromosome sequence, under Staphylococcus (Taxid: 1279) (Posted date: Aug. 5, 2008 4:57 AM). The NCBI BLAST Basic Local Alignment Search Tool (nucleotide Blast) was used to test the sequence similarities under three different conditions: 1) highly similar sequence; 2) more dissimilar sequence; and 3) somewhat similar sequence.

The sequence comparison results showed that there is no homology in either SEQ ID NO:1, SEQ ID NO:3, or SEQ ID NO:4 in any of aforementioned five DNA databases. No significant similarity was found even when the least stringent condition—somewhat similar sequence—was chosen as comparison criteria.

The blast result of SEQ ID NO:2 (from base pair number 2108819 to 2108960, GenBank accession number BA000033) against each of the aforementioned five DNA databases shows various hits according to the stringency of the condition (Table 1). The numbers in Table 1 indicate the blast hits.

TABLE 1
Result of blast hits of SEQ ID NO:
2 against various DNA databases

| | Highly | More | Somewhat |
|---|---|---|---|
| Database | similar | dissimilar | similar |
| Human genome | 0 | 0 | 58 |
| Patent sequence | 0 | 0 | 101 |
| NCBI chromosome | 3 (all are | 9 (three are | 147 |
| | Staphylococcus) | Staphylococcus) | |
| NCBI chromosome | 3 (all are | 3 (all are | 41 |
| (Bacteria, Taxid: 2) | Staphylococcus) | Staphylococcus) | |
| NCBI chromosome | 3 (all are | 3 (all are | 84 |
| (Staphylococcus, | Staphylococcus) | Staphylococcus) | |
| Taxid: 1279) | | | |

Various alignments based on the stringency of the condition are described below:

1) Alignment Under High Stringency Condition (Highly Similar):

SEQ ID NO:2 (from base pair number 2108819 to 2108960, GenBank accession number BA000033) does not align with any of sequences within human or patent sequence databases under most stringent condition (Table 1).

Results shown in Table 1 confirmed that SEQ ID NO:2 (from base pair number 2108819 to 2108960, GenBank accession number BA000033) is identical to the agrC corresponding sequence in another CA-MRSA MW2 strain (NC_{—}003923.1). This region is also 100% identical to an autoinducer sensor protein in a S. aureus strain MSSA476 (from base pair number 2087944 to 2088085, GenBank accession number BX571857.1). MSSA476 also caused severe invasive disease in the community and belonged to a major clone associated with community-acquired diseases that also contains the CA-MRSA strain MW2 (Enright et al., 2000, J. Clin. Microbiol. 38:1008-1015; Holden et al., 2004, PNAS 101:9786-9791).

Result shown in Table 1 also confirmed that SEQ ID NO:2 (from base pair number 2108819 to 2108960, GenBank accession number BA000033) is 98% identical to an autoinducer sensor protein in a S. aureus strain MRSA252 (from base pair number 2184837 to 2184978, GenBank accession number BX571856.1). MRSA252 belongs to the clinically important EMRSA-16 clone that is responsible for half of all MRSA infection in the U.K. (Johnson et al., 2001, J. Antimicrobiol. Chemother. 48:143-144), and is one of the major MRSA clones found in the U.S. (USA200) (McDougal et al., 2003, J. Clin. Microbiol. 41:5113-5120). Two mismatches found in this region occurred at the exact position when aligned to agr-3ₛₐ (GenBank accession number AF001783) corresponding sequence.

2) Alignment Under Less Stringent Condition (More Dissimilar):

SEQ ID NO:2 (from base pair number 2108819 to 2108960, GenBank accession number BA000033) do not align with any of sequences within human or patent sequence databases under less stringent condition (Table 1).

However, in addition to the homology with three S. aureus strains described above, alignment of SEQ ID NO:2 (from base pair number 2108819 to 2108960, GenBank accession number BA000033) against NCBI chromosome database picked up six sequences which spans SEQ ID NO:2 DNA region from 2108821 to 2108880 under less stringent condition. These six sequences consists of three sequences derived from Homo sapiens chromosome 11 (GenBank accession numbers AC_{—}000143.1, AC_{—}000054.1, and NC_{—}000011.8) with 78% homology; one sequence derived from Pan troglodytes chromosome 11 (GenBank accession numbers NC_{—}006478.2) with 78% homology; and two sequences derived from Rattus norvegicus chromosome 17 (GenBank accession numbers AC_{—}000085.1 and NC_{—}005112.2) with 84% homology.

3) Alignment Under the Least Stringent Condition (Somewhat Similar):

a) Search in Human Genome Database:

A total of 58 blast hits resulted when using SEQ ID NO:2 (from base pair number 2108819 to 2108960, GenBank accession number BA000033) as the query sequence to align human genome database under the least stringent condition (somewhat similar) (Table 1). The distribution of this 58 blast hits clusters mostly within two regions of SEQ ID NO:2—from base pair number 2108820 to base pair number 2108860 and from base pair number 2108898 to base pair number 2108949. The most non-homologous region within SEQ ID NO:2 located at two regions—from base pair number 2108861 to base pair number 2108897 and from base pair number 2108950 to base pair number 2108960.

b) Search in Patent Sequence Database:

A total of 101 blast hits resulted when using SEQ ID NO:2 (from base pair number 2108819 to 2108960, GenBank accession number BA000033) as the query sequence to align database in Patent Division of NCBI GenBank under the least stringent condition (somewhat similar) (Table 1). These blast hits aligned with DNA sequences that are random and short, and except one house mouse gene (GenBank accession number CS788875, 91% homology, spanning the region of SEQ ID NO:2 from base pair number 2108927 to base pair number 2108950), there are no sequence aligned beyond base pair number 2108947 in SEQ ID NO:2. The house mouse gene is unlikely to be mixed with human clinical samples designed to be tested in this invention. The most non-homologous region within SEQ ID NO:2 located at a region spanning base pair number 2108848 to base pair base pair number 2108960.

However, an artificial sequence of forward primer for a DNA macro-array analysis for agrC-MW2 gene was found in this alignment. This artificial sequence (GenBank accession number CS107636 and CS116412) spans the region corresponding to SEQ ID NO:2 from base pair number 2108928 to base pair number 2108947 with 100% homology. This region is not within the range for primer sequence design in this invention.

c) Search in NCBI Chromosome Database:

A total of 147 blast hits resulted when using SEQ ID NO:2 (from base pair number 2108819 to 2108960, GenBank accession number BA000033) as the query sequence to align NCBI chromosome database under the least stringent condition (somewhat similar) (Table 1). Excluding the homology in the three S. aureus strains, the distribution of the rest of 144 blast hits are random but there are only 6 hits with its 3′ end extended to the region corresponding to SEQ ID NO:2 base pair number 2108960. However, these 6 hits include two genes in mouse genome (GenBank accession number NC_{—}000078.5 and AC_{—}000034.1); one Equus caballus gene (GenBank accession number NC_{—}009148.2); two Rattus norvegicus genes (GenBank accession number NC_{—}005101.2 and AC_{—}000070.1); and one Canis familiaris gene (GenBank accession number NC_{—}006620.2). Those genes are unlikely to be mixed with human clinical samples designed to be tested in this invention. The most non-homologous region within SEQ ID NO:2 located at a region from base pair number 2108942 to base pair number 2108960.

d) Search in NCBI Chromosome (Bacteria Taxid: 2) Database:

A total of 41 blast hits resulted when using SEQ ID NO:2 (from base pair number 2108819 to 2108960, GenBank accession number BA000033) as the query sequence to align NCBI chromosome (bacteria Taxid: 2) database under the least stringent condition (somewhat similar) (Table 1). Excluding the homology in the three S. aureus strains, the distribution of the rest of 38 blast hits aligned with DNA sequences that are random and short. Among them, only 3 hits have its 3′ end extended to the region corresponding to SEQ ID NO:2 base pair number 2108952. These 3 hits include two genes in Leptospira interrogans genome (GenBank accession number NC_{—}004342.1 and NC_{—}005823.1) with 82% homology, spanning the region of SEQ ID NO:2 from base pair number 2108914 to base pair number 2108952, and one gene in Clostridium botulinum (GenBank accession number NC 010723.1) with 87% homology, spanning the region of SEQ ID NO:2 from base pair number 2108920 to base pair number 2108950. The most non-homologous region within SEQ ID NO:2 located at a region from base pair number 2108952 to base pair number 2108960.

e) Search in NCBI Chromosome (Staphylococcus Taxid: 1279) Database:

A total of 84 blast hits resulted when using SEQ ID NO:2 (from base pair number 2108819 to 2108960, GenBank accession number BA000033) as the query sequence to align NCBI chromosome (Staphylococcus Taxid: 1279) database under the least stringent condition (somewhat similar) (Table 1). Excluding the homology in the three S. aureus strains, the distribution of the rest of 81 blast hits clustered mostly within two regions of SEQ ID NO:2—from base pair number 2108835 to base pair number 2108863 and from base pair number 2108870 to base pair number 2108938. These two regions aligned with various S. aureus strains including S. aureus MSSA476 (GenBank accession number NC_{—}002953.3), S. aureus MW2 (GenBank accession number NC_{—}003923.1), S. aureus MRSA252 (GenBank accession number NC_{—}002952.2), S. aureus USA300_TCH1516 (GenBank accession number NC_{—}010079.1), S. aureus Mu3 (GenBank accession number NC_{—}009782.1), S. aureus Newman (GenBank accession number NC_{—}009641.1), S. aureus JH1 (GenBank accession number NC_{—}009632.1), S. aureus JH9 (GenBank accession number NC_{—}009487.1), S. aureus NCTC 8325 (GenBank accession number NC_{—}007795.1), S. aureus USA300 FPR3757 (GenBank accession number NC_{—}007793.1), S. aureus N315 (GenBank accession number NC_{—}002745.2), S. aureus Mu50 (GenBank accession number NC_{—}002758.2), and S. aureus COL (GenBank accession number NC_{—}002951.2).

The alignment pick up one Staphylococcus saprophyticus ATCC15305 gene (GenBank accession number NC_{—}007350.1) with 88% homology, spanning the region of SEQ ID NO:2 from base pair number 2108875 to base pair number 2108899.

Three sequences in Staphylococcus haemolyticus JCSC1435 (GenBank accession number NC_{—}007168.1) are also aligned with SEQ ID NO:2. These three sequences correspond to SEQ ID NO:2 from base pair number 2108886 to base pair number 2108904 (94% homology); from base pair number 2108914 to base pair number 2108938 (88% homology); and from base pair number 2108911 to base pair number 2108929 (94% homology).

Four sequences in Staphylococcus epidermidis ATCC 12228 (GenBank accession number NC_{—}004461.1) are also aligned with SEQ ID NO:2. These four sequences correspond to SEQ ID NO:2 from base pair number 2108837 to base pair number 2108853 (100% homology), from base pair number 2108898 to base pair number 2108914 (100% homology), from base pair number 2108830 to base pair number 2108850 (90% homology), and from base pair number 2108835 to base pair number 2108884 (74% homology).

The most non-homologous region within SEQ ID NO:2 located at two regions—from base pair number 2108861 to base pair number 2108869 and from base pair number 2108939 to base pair number 2108960.

FIG. 1 Step 30: designing primer sets for amplifying specific agr gene sequence in CA-MRSA MW2 or CA-MRSA USA300 strains

a) Primer Set to Amplify SEQ ID NO:1:

Because SEQ ID NO:1 is unique with no significant similarity found in all GenBank search performed, the following primer set was designed to amplified this DNA fragment:

(SEQ ID NO: 5)

forward primer 5′TTTTTATAATAATTTTTCTC3′

(SEQ ID NO: 6)

reverse primer 5′CTAAAAATACAGAATATAGT3′

b) Primer Set to Amplify SEQ ID NO:2:

Based on FIG. 1 Step 20 1), 2) and 3), the following primer set was selected for amplifying SEQ ID NO:2 at the same time avoiding co-amplifying genes in human, bacteria or Staphylococcus sp. other then S. aureus strains:

(SEQ ID NO: 7)

forward primer 5′TTTATTAGTGGAATAAGAT3′

(SEQ ID NO: 8)

reverse primer 5′TTGTTATAACAACTAGTGA3′

c) Primer Set to Amplify SEQ ID NO:3:

Because SEQ ID NO:3 is unique with no significant similarity found in all GenBank search performed, the following primer set was designed to amplified this 65 base pair DNA fragment:

(SEQ ID NO: 9)

forward primer 5′ATTTATACTTTTACCTTTA3′

(SEQ ID NO: 10)

reverse primer 5′TTAAAATAATCATAATTAAA3′

d) Primer Set to Amplify SEQ ID NO:4:

Because SEQ ID NO:4 is unique with no significant similarity found in all GenBank search performed, the following primer set was designed to amplified this 66 base pair DNA fragment:

(SEQ ID NO: 11)

forward primer 5′TATTCTTTTATTTTTATTGGT3′

(SEQ ID NO: 12)

reverse primer 5′AAATTGAGAAATAACAAATGTT3′

Synthesis of these oligonucleotides can easily be done by commercial companies, such as Integrated DNA technologies (Coralville, Iowa).

FIG. 1 Step 40: labeling the agr primer sets at 5′ end with a fluorescent reporter attached methylisocytosine (iso-dC), or labeling with other labeling substance

A newly developed Plexor system (Promega Corporation, Madison, Wis.) is applied to label a primer at 5′ end with a modified nucleotide (iso-dC) and a fluorophore. The selection of a fluorophore attached to each of the primer is based on the combination of the multiplex PCR amplification desired, as well as the detection capabilities of the real-time instrument used. The instruments currently available, with the compatibility to install the Plexor™ Analysis Software to visualize amplification data, plot standard curves and calculate DNA concentrations of unknowns, include ABI PRISM® 7000 and 7700 sequence detection systems, Applied Biosystems 7300, 7500 and 7900HT real time PCR system, Roche LightCycler® 1.0 and 2.0 instruments, Bio-Rad iCycler® thermal cycler, MJ Research DNA Engine Opticon® 2 fluorescence detection system, Cepheid SmartCycler® system and the Stratagene real-time PCR systems.

For example, if the available instrument is a Applied Biosystems 7500 real time PCR system (Forster City, Calif.) with 520 nm, 550 nm, 580 nm, 610 nm, and 650 nm filters installed, then the primers can be labeled in the order of use as FAM™, HEX™, Cal Fluor® Red 610, and Cy5™. The labeled primer can be synthesized by commercial companies such as Promega or Bioresearch Technologies.

To amplify and detect SEQ ID NO:1 and SEQ ID NO:2 for CA-MRSA MW2 strain, one can, for example, choose to label primers SEQ ID NO:5 and SEQ ID NO:7 with FAM™ and HEX™, respectively. Then, the remaining two filter channels are reserved for the multiplex PCR amplification for pvl and spa genes in one PCR reaction mixture.

Alternatively, to amplify and detect SEQ ID NO:3 and SEQ ID NO:4 for CA-MRSA USA300 strain in a separate reaction mixture, one can, for example, choose to label primers SEQ ID NO:9 and SEQ ID NO:11 with FAM™ and HEX™, respectively. Then, the remaining two filter channels are reserved for the multiplex PCR amplification for pvl and spa genes in one PCR reaction mixture.

A PCR reaction mixture can also be packaged into a combination of detecting CA-MRSA MW2 and USA300 strain in one tube by labeling SEQ ID NO:5 (for detecting SEQ ID NO:1 in MW2 strain) and SEQ ID NO:9 (for detecting SEQ ID NO:3 in USA300 strain) with FAM™ and HEX™, respectively. The remaining two filter channels are reserved for the multiplex PCR amplification for pvl and spa genes in one PCR reaction mixture.

Alternatively, if the purpose is only to type the agr gene (in the situation that the S. aureus status is already confirmed), then the combination of labeling SEQ ID NO:5 (for detecting SEQ ID NO:1 in CA-MRSA MW2 strain) and SEQ ID NO:7 (for detecting SEQ ID NO:2 in CA-MRSA MW2 strain) with FAM™ and HEX™, respectively, and SEQ ID NO:9 (for detecting SEQ ID NO:3 in CA-MRSA USA300 strain) and SEQ ID NO:11 (for detecting SEQ ID NO:4 in CA-MRSA USA300 strain) with Cal Fluor® Red 610 and Cy5™, respectively, can be packaged in one multiplex PCR mixture.

In addition to the labeling methods described above, a two-step process which uses T4 polynucleotide kinase to transfer a thiophosphate from ATPγS to the 5′-OH of the oligonucleotides, followed by coupling with a fluorochrome, biotin or affinity tag via a thio-reactive maleimide (Vector Labs, Burlingame, Calif.), can be utilized to label the aforementioned primer sets. For example, 5′ end biotin labeled primer can be applied in one amplicon PCR, and the PCR product can then be detected by conventional gel electrophoresis and chemiluminescence.

Although the primary goal for this invention is focused on primer sets and DNA sequences for a rapid identification of CA-MRSA MW2 or USA300 strain in clinical samples by multiplex PCR, the invention of the unique agr gene sequences that represents each strain and the primer sets to amplify them offers many other applications. For example, the agr primer sets and the agr gene sequence can also be used in identifying the causal agent in tissue samples by in situ hybridization. In this situation, one can label the primers with Fluorescein or Texas Red at 5′ end, or to combine the template DNA and the primer with a commercial available random primer labeling kit (such as described in USB, Cleveland, Ohio) to incorporate [α-³²P]-dATP or [α-³²P]-dCTP into a DNA probe.

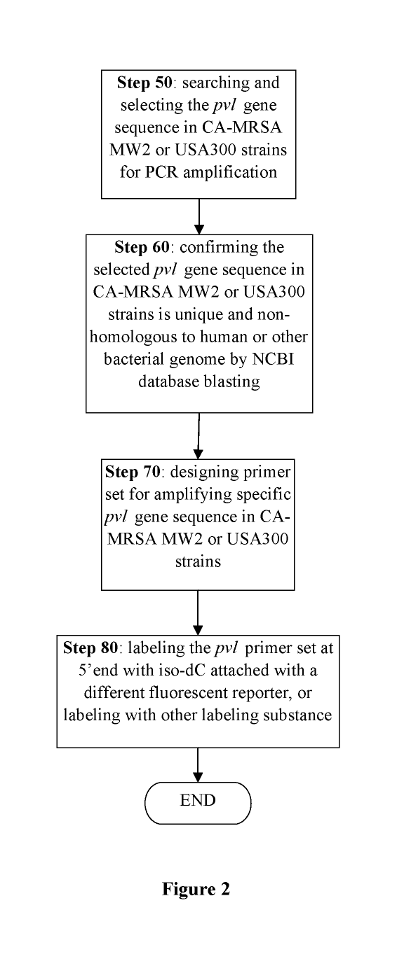

In the second dimension, this invention provides additional tool to amplify a partial pvl gene sequence in CA-MRSA MW2 or USA300 strains. A flow chart of steps that describe the invention of proper pvl gene and primer sequences is depicted in FIG. 2. Detailed description of FIG. 2 is presented by steps as follows.

FIG. 2 Step 50: Searching and selecting the pvl gene sequences in CA-MRSA MW2 or USA300 strains for PCR amplification

Based on the published sequences of pvl genes (GenBank accession number X72700, S. aureus ATCC 49775), a blast 2 search was performed to align corresponding pvl sequences in CA-MRSA MW2 and USA300. The result showed that pvl sequence in CA-MRSA MW2 spans the region from base pair number 1918488 to base pair number 1920484 (GenBank accession number BA000033), and in CA-MRSA USA300, it spans the region from base pair number 1955328 to base pair number 1957326 (GenBank accession number NC_{—}007793). The pvl sequence in CA-MRSA MW2 and USA300 shares a 99% homology. A region in CA-MRSA MW2 pvl sequence spanning base pair number 1919380 to base pair number 1919820 (GenBank accession number BA000033) was selected for PCR amplification and primer design.

FIG. 2 Step 60: confirming the selected pvl gene sequence in CA-MRSA MW2 or USA300 strains is unique and non-homologous to human or other bacterial genome by NCBI database blasting

To avoid co-amplification of human genome and other possible contaminants, a region in CA-MRSA MW2 pvl sequence spanning base pair number 1919380 to base pair number 1919820 (GenBank accession number BA000033) was used as a query sequence in a local alignment to search for the homology within several databases in NCBI.

a) Search in Human Genome Database:

A total of 96 blast hits resulted when using this CA-MRSA MW2 pvl sequence (from base pair number 1919380 to base pair number 1919820, GenBank accession number BA000033) as the query sequence to align human genome database under the least stringent condition (somewhat similar). However, there is no homology from base pair 1919380 to base pair number 1919429 and from base pair 1919740 to base pair number 1919820 (GenBank accession number BA000033).

b) Search in Patent Sequence Database:

A total of 102 blast hits resulted when using this CA-MRSA MW2 pvl sequence (from base pair number 1919380 to base pair number 1919820, GenBank accession number BA000033) as the query sequence to align patent sequence database under the least stringent condition (somewhat similar). The result shows that most of these 102 blast hits are S. aureus pvl sequence derived from various strains. However, none of these patented sequences are primer sequences.

c) Search in NCBI Chromosome Database:

A total of 151 blast hits resulted when using this CA-MRSA MW2 pvl sequence (from base pair number 1919380 to 1919820, GenBank accession number BA000033) as the query sequence to align NCBI chromosome database under the least stringent condition (somewhat similar). The result shows that a total of 13 blast hits are S. aureus pvl sequence from various S. aureus strains which spans the whole region with the homology ranging from 100% to 98%. These S. aureus strains include S. aureus USA300_TCH1516 (GenBank accession number NC_{—}010079.1), S. aureus Newman (GenBank accession number NC_{—}009641.1), S. aureus NCTC 8325 (GenBank accession number NC_{—}007795.1), S. aureus USA300 (GenBank accession number NC_{—}007793.1), S. aureus COL (GenBank accession number NC_{—}002951.2), S. aureus MSSA476 (GenBank accession number NC_{—}002953.3), S. aureus MW2 (GenBank accession number NC_{—}003923.1), S. aureus Mu3 (GenBank accession number NC_{—}009782.1), S. aureus JH1 (GenBank accession number NC_{—}009632.1), S. aureus JH9 (GenBank accession number NC_{—}009487.1), S. aureus N315 (GenBank accession number NC_{—}002745.2), S. aureus Mu50 (GenBank accession number NC_{—}002758.2), and. S. aureus RF122 (GenBank accession number NC_{—}007622.1).

The rest of the blast hits aligned with much shorter DNA regions with various organisms. However, there is no homology aligned to CA-MRSA MW2 pvl sequence from base pair number 1919380 to base pair number 1919395 and from base pair base pair number 1919749 to base pair number 1919820 (GenBank accession number BA000033).

d) Search in NCBI Chromosome (Bacteria Taxid: 2) Database:

A total of 87 blast hits resulted when using this CA-MRSA MW2 pvl sequence (from base pair number 1919380 to base pair number 1919820, GenBank accession number BA000033) as the query sequence to align NCBI chromosome (bacteria Taxid: 2) database under the least stringent condition (somewhat similar). The result also shows that a total of 13 blast hits are S. aureus pvl sequence from various S. aureus strains which spans the whole region with the homology ranging from 100% to 98%. These S. aureus strains include S. aureus USA300_TCH1516 (GenBank accession number NC_{—}010079.1), S. aureus Newman (GenBank accession number NC_{—}009641.1), S. aureus NCTC 8325 (GenBank accession number NC_{—}007795.1), S. aureus USA300 (GenBank accession number NC_{—}007793.1), S. aureus COL (GenBank accession number NC_{—}002951.2), S. aureus MSSA476 (GenBank accession number NC_{—}002953.3), S. aureus MW2 (GenBank accession number NC_{—}003923.1), S. aureus Mu3 (GenBank accession number NC_{—}009782.1), S. aureus JH1 (GenBank accession number NC_{—}009632.1), S. aureus JH9 (GenBank accession number NC_{—}009487.1), S. aureus N315 (GenBank accession number NC_{—}002745.2), S. aureus Mu50 (GenBank accession number NC_{—}002758.2), and. S. aureus RF122 (GenBank accession number NC_{—}007622.1).

The rest of the blast hits aligned with much shorter DNA regions with various organisms. Except for one hit, there is no homology aligned to CA-MRSA MW2 pvl sequence from base pair number 1919380 to base pair number 1919404 and from base pair number 1919786 to base pair number 1919820 (GenBank accession number BA000033). The only one exception is a DNA sequence encoded a putative type II restriction enzyme Sau3AI by Bifidobacterium adolescentis ATCC 15703 (GenBank accession number NC_{—}008618.1) which shares 84% homology with CA-MRSA MW2 pvl sequence from base pair number 1919391 to base pair number 1919423 (GenBank accession number BA000033). B. adolescentis is not a human pathogen, and is unlikely to be contaminated with human clinical samples.

e) Search in NCBI Chromosome (Staphylococcus Taxid: 1279) Database:

A total of 131 blast hits resulted when using CA-MRSA MW2 pvl sequence (from base pair number 1919380 to base pair number 1919820, GenBank accession number BA000033) as the query sequence to align NCBI chromosome (Staphylococcus Taxid: 1279) database under the least stringent condition (somewhat similar).

The result also shows that a total of 13 blast hits are S. aureus pvl sequence from various S. aureus strains which spans the whole region with the homology ranging from 100% to 98%. These S. aureus strains include S. aureus USA300_TCH1516 (GenBank accession number NC_{—}010079.1), S. aureus Newman (GenBank accession number NC_{—}009641.1), S. aureus NCTC 8325 (GenBank accession number NC_{—}007795.1), S. aureus USA300 (GenBank accession number NC_{—}007793.1), S. aureus COL (GenBank accession number NC_{—}002951.2), S. aureus MSSA476 (GenBank accession number NC_{—}002953.3), S. aureus MW2 (GenBank accession number NC_{—}003923.1), S. aureus Mu3 (GenBank accession number NC_{—}009782.1), S. aureus JH1 (GenBank accession number NC_{—}009632.1), S. aureus JH9 (GenBank accession number NC_{—}009487.1), S. aureus N315 (GenBank accession number NC_{—}002745.2), S. aureus Mu50 (GenBank accession number NC_{—}002758.2), and. S. aureus RF122 (GenBank accession number NC_{—}007622.1).

The rest of the blast hits aligned with much shorter DNA regions in several coagulase negative Staphylococcus including S. haemolyticus JCSC1435 (GenBank accession number NC_{—}007168.1), S. saprophyticus ATCC15305 (GenBank accession number NC_{—}007350.1), and S. epidermidis RP62A (GenBank accession number NC_{—}002976.3). However, none of the homologous regions aligned with CA-MRSA MW2 pvl sequence from base pair number 1919380 to base pair number 1919407 and from base pair number 1919560 to base pair number 1919820 (GenBank accession number BA000033).

FIG. 2 Step 70: designing primer set for amplifying specific pvl gene sequence in CA-MRSA MW2 or USA300 strains.

Based on the FIG. 2 Step 60 a), b), c), d) and e), the following primer set was selected for amplifying pvl gene at the same time avoiding co-amplifying human and other bacterial sequences:

(SEQ ID NO: 13)

forward primer 5′CAGGTGTGATATGTTGAGCT3′

(SEQ ID NO: 14)

reverse primer 5′TATTTATTCGTACAAAGTCCA3′

Synthesis of these oligonucleotides can easily be done by commercial companies, such as Integrated DNA technologies (Coralville, Iowa).

FIG. 2 Step 80: labeling the pvl primer set at 5′end with a fluorescent reporter attached iso-dC, or labeling with other labeling substance

A newly developed Plexor system (Promega Corporation, Madison, Wis.) as described in FIG. 1 Step 40 is applied to label a primer at 5′ end with a modified nucleotide (iso-dC) and a fluorophore. The selection of a fluorophore attached to the primer is based on the combination of the multiplex PCR amplification desired, as well as the detection capabilities of the real-time instrument used as described in FIG. 1 Step 40.

For example, as described in FIG. 1 Step 40, if the available instrument is a Applied Biosystems 7500 real time PCR system (Forster City, Calif.) with 520 nm, 550 nm, 580 nm, 610 nm, and 650 nm filters installed, then the primers can be labeled in the order of use as FAM™, HEX™, Cal Fluor® Red 610, and Cy5™. The labeled primer can be synthesized by commercial companies such as Promega (Madison, Wis.).

As described in FIG. 1 Step 40, FAM™ and HEX™ may be used to label primers SEQ ID NO:5 and SEQ ID NO:7 to amplify and detect SEQ ID NO:1 and SEQ ID NO:2 for CA-MRSA MW2 strain. In this case, one can choose to label SEQ ID NO:13 with Cal Fluor® Red 610 for the detection of pvl gene as an example.

As described in FIG. 1 Step 40, other labeling methods, such as coupling with a fluorochrome, biotin, or affinity tag at 5′ end of the primer, can also be applied for one amplicon PCR or in situ hybridization.

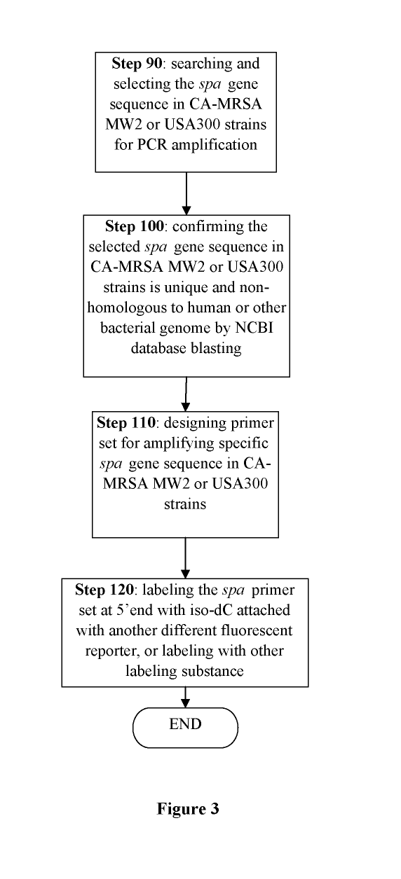

In the third dimension, this invention provides additional tool to amplify a partial spa gene sequence in CA-MRSA MW2 or USA300 strains. A flow chart of steps that describe the invention of proper spa gene and primer sequences is depicted in FIG. 3. Detailed description of FIG. 3 is presented by steps as follows.

FIG. 3 Step 90: searching and selecting the spa gene sequences in CA-MRSA MW2 or USA300 strains for PCR amplification

A blast 2 search was performed, based on the published sequences of spa gene (GenBank accession number J01786, S. aureus strain 8325-4, Uhlen et al., 1984, JBC 259:1695-1702), to align corresponding spa sequences in CA-MRSA MW2 and USA300. A short DNA region, spanning spa gene sequence in CA-MRSA MW2 from base pair number 99612 to base pair number 99792 (GenBank accession number BA000033) was selected for PCR amplification and primer design. The spa sequences in CA-MRSA MW2 and USA300 share a 100% homology in this region.

FIG. 3 Step 100: confirming the selected spa gene sequence in CA-MRSA MW2 or USA300 strains is unique and non-homologous to human or other bacterial genome by NCBI database blasting

To avoid co-amplification of human genome and other possible contaminants, a region in CA-MRSA MW2 spa sequence spanning base pair number 99652 to base pair number 99792 (GenBank accession number BA000033) was used as a query sequence in a local alignment to search for the homology within several databases in NCBI.

a) Search in Human Genome Database:

A total of 22 blast hits resulted when using this CA-MRSA MW2 spa sequence (from base pair number 99652 to base pair number 99792, GenBank accession number BA000033) as the query sequence to align human genome database under the least stringent condition (somewhat similar). However, there is no homology from base pair number 99652 to base pair number 99664 and from base pair 99750 to base pair number 99767 (GenBank accession number BA000033).

b) Search in Patent Sequence Database:

A total of 100 blast hits resulted when using this CA-MRSA MW2 spa sequence (from base pair number 99652 to base pair number 99792, GenBank accession number BA000033) as the query sequence to align patent sequence database under the least stringent condition (somewhat similar). The result shows that many of these 100 blast hits are S. aureus spa sequence, however, none of these patented sequences are primer sequences.

c) Search in NCBI Chromosome Database:

A total of 54 blast hits resulted when using this CA-MRSA MW2 spa sequence (from base pair number 99652 to base pair number 99792, GenBank accession number BA000033) as the query sequence to align NCBI chromosome database under the least stringent condition (somewhat similar). The result shows that a total of 14 blast hits are S. aureus spa sequence from various S. aureus strains which spans the whole region with the homology ranging from 100% to 90%. These S. aureus strains include S. aureus USA300_TCH1516 (GenBank accession number NC_{—}010079.1), S. aureus Newman (GenBank accession number NC_{—}009641.1), S. aureus NCTC 8325 (GenBank accession number NC_{—}007795.1), S. aureus USA300 (GenBank accession number NC_{—}007793.1), S. aureus COL (GenBank accession number NC_{—}002951.2), S. aureus MSSA476 (GenBank accession number NC_{—}002953.3), S. aureus MW2 (GenBank accession number NC_{—}003923.1), S. aureus Mu3 (GenBank accession number NC_{—}009782.1), S. aureus JH1 (GenBank accession number NC_{—}009632.1), S. aureus JH9 (GenBank accession number NC_{—}009487.1), S. aureus N315 (GenBank accession number NC_{—}002745.2), S. aureus Mu50 (GenBank accession number NC_{—}002758.2), S. aureus MRSA252 (GenBank accession number NC_{—}002952.2), and S. aureus RF122 (GenBank accession number NC_{—}007622.1).

The rest of the blast hits aligned with much shorter DNA regions with various organisms. These DNA regions are too short and unlikely to be amplified when CA-MRSA MW2 spa sequence from base pair number 99652 to base pair number 99664 and from base pair number 99750 to base pair number 99767 (GenBank accession number BA000033) are used as primer sets for PCR.

d) Search in NCBI Chromosome (Bacteria Taxid: 2) Database:

A total of 70 blast hits resulted when using this CA-MRSA MW2 spa sequence (from base pair number 99651 to base pair number 99792, GenBank accession number BA000033) as the query sequence to align NCBI chromosome (bacteria Taxid: 2) database under the least stringent condition (somewhat similar). The result also shows that a total of 14 blast hits are S. aureus spa sequence from various S. aureus strains which spans the whole region with the homology ranging from 100% to 99%. These S. aureus strains include S. aureus USA300_TCH1516 (GenBank accession number NC_{—}010079.1), S. aureus Newman (GenBank accession number NC_{—}009641.1), S. aureus NCTC 8325 (GenBank accession number NC_{—}007795.1), S. aureus USA300 (GenBank accession number NC_{—}007793.1), S. aureus COL (GenBank accession number NC_{—}002951.2), S. aureus MSSA476 (GenBank accession number NC_{—}002953.3), S. aureus MW2 (GenBank accession number NC_{—}003923.1), S. aureus Mu3 (GenBank accession number NC_{—}009782.1), S. aureus JH1 (GenBank accession number NC_{—}009632.1), S. aureus JH9 (GenBank accession number NC_{—}009487.1), S. aureus N315 (GenBank accession number NC_{—}002745.2), S. aureus Mu50 (GenBank accession number NC_{—}002758.2), S. aureus MRSA252 (GenBank accession number NC_{—}002952.2) and S. aureus RF122 (GenBank accession number NC_{—}007622.1).

The rest of the blast hits aligned with much shorter DNA regions with various organisms. These DNA regions are too short and unlikely to be amplified when CA-MRSA MW2 spa sequence from base pair number 99652 to base pair number 99664 and from base pair 99750 to base pair number 99767 (GenBank accession number BA000033) are used as primer sets for PCR.

e) Search in NCBI Chromosome (Staphylococcus Taxid: 1279) Database:

A total of 74 blast hits resulted when using CA-MRSA MW2 spa sequence (from base pair number 99651 to base pair number 99792, GenBank accession number BA000033) as the query sequence to align NCBI chromosome (Staphylococcus Taxid: 1279) database under the least stringent condition (somewhat similar).

The result also shows that a total of 14 blast hits are S. aureus spa sequence from various S. aureus strains which spans the whole region with the homology ranging from 100% to 99%. These S. aureus strains include S. aureus USA300_TCH1516 (GenBank accession number NC_{—}010079.1), S. aureus Newman (GenBank accession number NC_{—}009641.1), S. aureus NCTC 8325 (GenBank accession number NC_{—}007795.1), S. aureus USA300 (GenBank accession number NC_{—}007793.1), S. aureus COL (GenBank accession number NC_{—}002951.2), S. aureus MSSA476 (GenBank accession number NC_{—}002953.3), S. aureus MW2 (GenBank accession number NC_{—}003923.1), S. aureus Mu3 (GenBank accession number NC_{—}009782.1), S. aureus JH1 (GenBank accession number NC_{—}009632.1), S. aureus JH9 (GenBank accession number NC_{—}009487.1), S. aureus N315 (GenBank accession number NC_{—}002745.2), S. aureus Mu50 (GenBank accession number NC_{—}002758.2), S. aureus MRSA252 (GenBank accession number NC_{—}002952.2) and S. aureus RF122 (GenBank accession number NC_{—}007622.1).

The rest of the blast hits aligned with much shorter DNA regions in several coagulase negative Staphylococcus including S. haemolyticus JCSC1435 (GenBank accession number NC_{—}007168.1), S. saprophyticus ATCC15305 (GenBank accession number NC_{—}007350.1), S. epidermidis ATCC12228 (GenBank accession number NC_{—}004461.1) and S. epidermidis RP62A (GenBank accession number NC_{—}002976.3). However, these DNA regions are too short and unlikely to be amplified when MW2 spa sequence from base pair number 99652 to base pair number 99664 and from base pair number 99750 to base pair number 99767 (GenBank accession number BA000033) are used as primer sets for PCR.

FIG. 3 Step 110: designing primer sets for amplifying specific spa gene sequence in CA-MRSA MW2 or USA300 strains

Based on the FIG. 3 Step 100 a), b), c), d), and e), the following primer set was selected for amplifying spa gene at the same time avoiding co-amplifying human and other bacterial sequences:

(SEQ ID NO: 15)

forward primer 5′TCAACAACAAGTTCTTGACC3′

(SEQ ID NO: 16)

reverse primer 5′ACAGTAAATGACATTGCAAAA3′

Synthesis of these oligonucleotides can easily be done by commercial companies, such as Integrated DNA technologies (Coralville, Iowa).

FIG. 3 Step 120: labeling the spa primer set at 5′end with a fluorescent reporter attached iso-dC, or labeling with other labeling substance

A newly developed Plexor system (Promega Corporation, Madison, Wis.) as described in FIG. 1 Step 40 and FIG. 2 Step 80 is applied to label a primer at 5′ end with a modified nucleotide (iso-dC) and a fluorophore. The selection of a fluorophore attached to the primer is based on the combination of the multiplex PCR amplification desired, as well as the detection capabilities of the real-time instrument used as described in FIG. 1 Step 40 and FIG. 2 Step 80.

For example, as described in FIG. 1 Step 40 and FIG. 2 Step 80, if the available instrument is a Applied Biosystems 7500 real time PCR system (Forster City, Calif.) with 520 nm, 550 nm, 580 nm, 610 nm, and 650 nm filters installed, then the primers can be labeled in the order of use as FAM™, HEX™, Cal Fluor® Red 610, and Cy5™. The labeled primer can be synthesized by commercial companies such as Promega (Madison, Wis.).

As described in FIG. 1 Step 40 and FIG. 2 Step 80, FAM™, HEX™ and Cal Fluor® Red 610 may be used to label primers SEQ ID NO:5, SEQ ID NO:7 and SEQ ID NO:13, respectively, to amplify and detect SEQ ID NO:1 and SEQ ID NO:2 for CA-MRSA MW2 strain and pvl gene. In this case, one can choose to label SEQ ID NO:15 with Cy5™ and package them in one multiplex PCR amplification mixture for the detection of spa gene.

As described in FIG. 1 Step 40 and FIG. 2 Step 80, other labeling methods, such as coupling with a fluorochrome, biotin, or affinity tag at 5′ end of the primer, can also be applied for one amplicon PCR or in situ hybridization.

What is claimed is:

1. A DNA molecule or an oligonucleotide primer comprising:
a) a part of or an entire nucleic acid sequence of the DNA molecule described in SEQ ID NO:1 or SEQ ID NO:2 (where A represents adenine, C represents cytosine, G represents guanine and T represents thymine, and T in any position may be replaced by uracil (U), and hereinafter the same abbreviations will be used), or a part of or an entire sequence of the complementary sequence thereof, wherein said SEQ ID NO:1 and SEQ ID NO:2 correspond to unique agr gene sequences in community-acquired methicillin-resistant Staphylococcus aureus (CA-MRSA) MW2 strain;
b) a part of or an entire nucleic acid sequence of the DNA molecule described in SEQ ID NO:3 or SEQ ID NO:4, or a part of or an entire sequence of the complementary sequence thereof, wherein said SEQ ID NO:3 and SEQ ID NO:4 correspond to unique agr gene sequences in CA-MRSA USA300 strain; and
c) a part of or an entire nucleic acid sequence of an oligonucleotide primer described in SEQ
ID NO:5, SEQ ID NO:6, SEQ ID NO:7, SEQ ID NO:8, SEQ ID NO:9, SEQ ID NO:10, SEQ ID NO:11 or SEQ ID NO:12, or a part of or an entire sequence of the complementary sequence thereof, wherein said oligonucleotide primer further comprises primer sets capable of amplifying or detecting said SEQ ID NO:1, SEQ ID NO:2, SEQ ID NO:3 or SEQ ID NO:4 individually or in combinations in the same strain.

2. A primer set according to claim 1, wherein the primer set comprises a forward primer having the sequence of SEQ ID NO:5 and a reverse primer having the sequence of SEQ ID NO:6 for amplifying or detecting said SEQ ID NO:1.

3. The primer set according to claim 2, wherein either the forward primer or the reverse primer is conjugated with methylisocytosine (iso-dC) to the 5′ end and then further labeled with a plurality of fluorophore adjacent to the iso-dC.

4. The primer set according to claim 2, wherein both the forward primer and the reverse primer are labeled with a labeling substance comprising of a radioisotope, an enzyme, a fluorescent substance, a luminescent substance, or a biotin.

5. A primer set according to claim 1, wherein the primer set comprises a forward primer having the sequence of SEQ ID NO:7 and a reverse primer having the sequence of SEQ ID NO:8 for amplifying or detecting said SEQ ID NO:2.

6. The primer set according to claim 5, wherein either the forward primer or the reverse primer is conjugated with methylisocytosine (iso-dC) to the 5′ end and then further labeled with a plurality of fluorophore adjacent to the iso-dC.

7. The primer set according to claim 5, wherein both the forward primer and the reverse primer are labeled with a labeling substance comprising of a radioisotope, an enzyme, a fluorescent substance, a luminescent substance, or a biotin.

8. A primer set according to claim 1, wherein the primer set comprises a forward primer having the sequence of SEQ ID NO:9 and a reverse primer having the sequence of SEQ ID NO:10 for amplifying or detecting said SEQ ID NO:3.

9. The primer set according to claim 8, wherein either the forward primer or the reverse primer is conjugated with methylisocytosine (iso-dC) to the 5′ end and then further labeled with a plurality of fluorophore adjacent to the iso-dC.

10. The primer set according to claim 8, wherein both the forward primer and the reverse primer are labeled with a labeling substance comprising of a radioisotope, an enzyme, a fluorescent substance, a luminescent substance, or a biotin.

11. A primer set according to claim 1, wherein the primer set comprises a forward primer having the sequence of SEQ ID NO:11 and a reverse primer having the sequence of SEQ ID NO:12 for amplifying or detecting said SEQ ID NO:4.

12. The primer set according to claim 11, wherein either the forward primer or the reverse primer is conjugated with methylisocytosine (iso-dC) to the 5′ end and then further labeled with a plurality of fluorophore adjacent to the iso-dC.

13. The primer set according to claim 11, wherein both the forward primer and the reverse primer are labeled with a labeling substance comprising of a radioisotope, an enzyme, a fluorescent substance, a luminescent substance, or a biotin.

14. A primer set for amplifying or detecting a partial S. aureus pvl gene comprising of a forward primer having the sequence of SEQ ID NO:13 and a reverse primer having the sequence of SEQ ID NO:14.

15. The primer set according to claim 14, wherein either the forward primer or the reverse primer is conjugated with methylisocytosine (iso-dC) to the 5′ end and then further labeled with a plurality of fluorophore adjacent to the iso-dC.

16. The primer set according to claim 14, wherein both the forward primer and the reverse primer are labeled with a labeling substance comprising of a radioisotope, an enzyme, a fluorescent substance, a luminescent substance, or a biotin.

17. A primer set for amplifying or detecting a partial S. aureus spa gene comprising of a forward primer having the sequence of SEQ ID NO:15 and a reverse primer having the sequence of SEQ ID NO:16.

18. The primer set according to claim 17, wherein either the forward primer or the reverse primer is conjugated with methylisocytosine (iso-dC) to the 5′ end and then further labeled with a plurality of fluorophore adjacent to the iso-dC.

19. The primer set according to claim 17, wherein both the forward primer and the reverse primer are labeled with a labeling substance comprising of a radioisotope, an enzyme, a fluorescent substance, a luminescent substance, or a biotin.
